(19) United States
(12) Patent Application Publication
(10) Pub. No.: US 2022/0323525 A1
(43) Pub. Date: Oct. 13, 2022

(54) FUKE QIANJIN TABLET AND QUALITY CONTROL METHOD THEREFOR

(71) Applicant: QIANJIN PHARMACEUTICAL CO., LTD., Hunan (CN)

(72) Inventors: Duanyu JIANG, Hunan (CN); Peng ZHANG, Hunan (CN); Yun GONG, Hunan (CN); Fujun LI, Hunan (CN); Yingshuai ZHANG, Hunan (CN); Lu BAI, Hunan (CN); Wei ZHAO, Hunan (CN); Kanghua WANG, Hunan (CN); Xiuwei YANG, Hunan (CN)

(73) Assignee: QIANJIN PHARMACEUTICAL CO., LTD., Hunan (CN)

(21) Appl. No.: 17/293,921

(22) PCT Filed: Jan. 14, 2020
(86) PCT No.: PCT/CN2020/072102

§ 371 Date: Jan. 14, 2022

(30) Foreign Application Priority Data
Nov. 14, 2018 (CN) 201811356102.1
Nov. 14, 2018 (CN) 201811356212.8

(51) Int. Cl. A61K 36/232; A61K 36/738; A61K 36/344; A61K 36/758; A61K 9/16; G01N 30/02

(52) U.S. Cl. CPC A61K 36/232; A61K 36/738; A61K 36/344; A61K 36/758; A61K 9/1694; G01N 30/02; G01N 2030/3007

(57) ABSTRACT

The present invention discloses Fuke Qianjin Tablets and a quality control method therefor. The Fuke Qianjin Tablets are made of Radix Et Caulis Flemingiae, Caulis Mahoniae, Herba Andrographis, Zanthoxylum dissitum Hemsl., Caulis Spatholobi, Radix Angelicae Sinensis, Radix Codonopsis, and Radix Rosa Laevigata as raw materials. Each of the Fuke Qianjin Tablets contains not less than 0.008 mg of the genistin, not less than 0.7 mg of the Z-ligustilide, and the total amount of the andrographolide and the dehydroandrographolide is not less than 1.1 mg.

### TECHNICAL FIELD

The present invention relates to the technical field of traditional Chinese medicine, and in particular, to Fuke Qianjin Tablets and a quality control method therefor.

### RELATED ART

Fuke Qianjin Tablet is a medicine made from 8 medicinal materials of Radix Et Caulis Flemingiae, Radix Rosa Laevigata, Herba Andrographis, Caulis Mahoniae, Zanthoxylum dissitum Hemsl., Radix Angelicae Sinensis, Caulis Spatholobi, and Radix Codonopsis. Its effect is to clear heat and eliminate dampness, and replenish and benefit qi and blood. It is used for leukorrheal diseases and abdominal pain caused by damp-heat stasis obstruction, symptoms of which include large quantity of leucorrhea, yellow in colour qualitative thick, and stinky, lower abdomen pain, lumbosacral soreness, and fatigued spirit and lack of strength; and chronic pelvic inflammatory disease, endometritis, and chronic cervicitis with the symptoms described above.

In the existing Pharmacopoeia of the People's Republic of China, for the content identification of the Fuke Qianjin Tablets, only total content of andrographolide and dehydroandrographolide is defined. Specifically, in each tablet, based on the total amount of andrographolide (C₂₀H₃₀O₅) and dehydroandrographolide (C₂₀H₂₈O₄), the content of Herba Andrographis shall not be less than 0.8 mg. Among compatible ingredients of the Fuke Qianjin Tablets, Herba Andrographis is only used as the minister drug, and obviously, the existing standards do not regulate other important ingredients in the Fuke Qianjin Tablets, so that it is difficult to control the consistency of effects between different batches of products of the Fuke Qianjin Tablets. Although relatively consistent products have been obtained through formulas and preparation methods, a more effective method is still needed to improve the therapeutic effect of the Fuke Qianjin Tablets.

### SUMMARY OF INVENTION

The technical problem to be solved by the present invention is to overcome the above-mentioned shortcomings and deficiencies of the prior art, to provide Fuke Qianjin Tablets and a quality control method therefor. Compared with the existing Fuke Qianjin Tablets, the Fuke Qianjin Tablets prepared by the quality control method of the present invention are more stable in terms of effect consistency, and have a better clinical treatment effect than the existing Fuke Qianjin Tablets.

An objective of the present invention is to provide a quality control method for Fuke Qianjin Tablets.

Another objective of the present invention is to provide Fuke Qianjin Tablets.

The above-described objectives of the present invention are realized by the following technical solutions.

A quality control method for Fuke Qianjin Tablets, including the following steps:

using Radix Et Caulis Flemingiae, Radix Rosa Laevigata, Herba Andrographis, Radix Angelicae Sinensis, Caulis Mahoniae, Zanthoxylum dissitum Hemsl., Caulis Spatholobi and Radix Codonopsis as raw materials;

S1, selecting the Radix Angelicae Sinensis, the Radix Codonopsis, and the Herba Andrographis, crushing into fine powders of 100 meshes or more, respectively, and reserving for use at a powder yield of at least 93.3%;

S2, selecting the Zanthoxylum dissitum Hemsl., the Radix Rosa Laevigata, the Caulis Spatholobi, Caulis Mahoniae and the Radix Et Caulis Flemingiae, adding water to extract twice, each extraction time being 2 hours, filtering to obtain a filtrate, and concentrating the filtrate into a cream of 1.08/85° CCF; and

S3, mixing the fine powders of the step S1 and the cream of the step S2 to obtain a mixture, controlling a content of genistin, a content of at least one of Z-ligustilide and Z-3-butylidenephthalide, and a total amount of andrographolide and dehydroandrographolide in the mixture to reach a standard content; and then drying, tabletting and coating to obtain the Fuke Qianjin Tablets.

In the Fuke Qianjin Tablets, the Radix Et Caulis Flemingiae and the Caulis Mahoniae clear heat and remove toxicity, eliminate dampness and arrest leucorrhoea, and together serve as a sovereign drug. The Herba Andrographis and the Zanthoxylum dissitum Hemsl. clear heat and remove toxicity, cool the blood and relieve swelling, eliminate dampness and arrest leucorrhoea, and serve as a minister drug. The Caulis Spatholobi and the Radix Angelicae Sinensis nourish the blood and promote blood circulation. The Radix Codonopsis benefits qi and strengthens spleen, and promotes transportation and digestion of water-dampness to arrest leucorrhoea. The Radix Rosa Laevigata arrests spontaneous emission and leucorrhoea, and serves as an assistant drug.

Traditional Chinese medicine compound is a hierarchical and structured organic whole. Its effect is not a simple addition of individual medicines, but a result of the mutual cooperation of multiple active ingredients. In the existing Pharmacopoeia, the standard regulations for the Fuke Qianjin Tablet only limit that andrographolide and dehydroandrographolide are from the minister drug (from Herba Andrographis), but do not limit types and contents of other active ingredients. In the actual production process, in quality detection and control, only andrographolide and dehydroandrographolide were detected, but types and contents of other active ingredients were not detected. As we all know, a content of an active ingredient in an extract of a traditional Chinese medicinal material is affected by the planting area of the medicinal material and the extraction method, so even if the extract is prepared according to the same raw material formula, a content of a specific active ingredient is not the same. Therefore, the content of other active ingredients that is not detected between different batches of the Fuke Qianjin Tablets are not uniform, resulting in large differences in their efficacy.

In order to ensure that the prepared Fuke Qianjin Tablets have the same efficacy in the actual production process, the inventors have found after many experiments that in addition to andrographolide and dehydroandrographolide, by controlling the contents of the three active ingredients of genistin, Z-ligustilide and Z-3-butylidenephthalide in the product within a certain range, the effect of the produced product is better, the efficacy of different batches is consistent, and the clinical treatment effect is improved. Due to the various influencing factors in a preparation process of a traditional Chinese medicine, the content control of core ingredients in the traditional Chinese medicine can overcome the problem of fluctuations in ingredient content between different batches. As a result, a new quality control method for the Fuke Qianjin Tablets is proposed. Using the quality control method, it can ensure that the contents of the five active ingredients in different batches of products are relatively consistent, and the consistency of the product's efficacy is relatively stable.

Preferably, in the step S3, it is controlled that per milligram of the mixture, the content of the genistin is not less than 0.00005 mg, the content of the Z-ligustilide is not less than 0.00394 mg and/or the content of the Z-3-butylidenephthalide is not less than 0.00008 mg, and the content of the andrographolide and the dehydroandrographolide is not less than 0.00619 mg.

More preferably, in the step S3, it is controlled that per milligram of the mixture, the content of the genistin is not less than 0.00007 mg, the content of the Z-ligustilide is not less than 0.0045 mg and/or the content of the Z-3-butylidenephthalide is not less than 0.00011 mg, and the content of the andrographolide and the dehydroandrographolide is not less than 0.00788 mg.

More preferably, in the step S3, it is controlled that per milligram of the mixture, the content of the genistin is 0.00011 mg to 0.00028 mg, the content of the Z-ligustilide is 0.00647 mg to 0.009 mg and/or the content of the Z-3-butylidenephthalide is 0.00023 mg to 0.00045 mg, and the content of the andrographolide and the dehydroandrographolide is not less than 0.01 mg.

Specifically and preferably, each of the Fuke Qianjin Tablets prepared by the method (according to the existing product and production index as a reference, the mass of each of the Fuke Qianjin Tablets is 0.32 g) contains not less than 0.008 mg of the genistin, not less than 0.7 mg of the Z-ligustilide and/or not less than 0.015 mg of the Z-3-butylidenephthalide, and the total amount of the andrographolide and the dehydroandrographolide is not less than 1.1 mg.

More preferably, each of the Fuke Qianjin Tablets prepared by the method (according to the existing product and production index as a reference, the mass of each of the Fuke Qianjin Tablets is 0.32 g) contains not less than 0.012 mg of the genistin, not less than 0.8 mg of the Z-ligustilide and/or not less than 0.02 mg of the Z-3-butylidenephthalidem, and the total amount of the andrographolide and the dehydroandrographolide is not less than 1.4 mg.

More preferably, in each of the Fuke Qianjin Tablets, the total amount of the andrographolide and the dehydroandrographolide is not less than 1.93 mg.

More preferably, each of the Fuke Qianjin Tablets prepared by the method (according to the existing product and production index as a reference, the mass of each of the Fuke Qianjin Tablets is 0.32 g) contains 0.02 mg to 0.05 mg of the genistin, 1.15 mg to 1.65 mg of the Z-ligustilide and/or 0.04 mg to 0.08 mg of the Z-3-butylidenephthalide, and the total amount of the andrographolide and the dehydroandrographolide is not less than 1.93 mg.

Preferably, in the step S3, said controlling makes the contents of the genistin, the Z-ligustilide and/or the Z-3-butylidenephthalide after the 4 creams in the steps S1 to S4 are mixed reach a required range by adjusting an extraction process of the step S2 or a source of the raw materials.

Preferably, the method of the step S2 is to select the Zanthoxylum dissitum Hemsl. And the Radix Rosa Laevigata, the Caulis Spatholobi, the Caulis Mahoniae and the Radix Et Caulis Flemingiae, to add water to extract twice, wherein adding water that is 10 times a total weight of the 5 traditional Chinese medicine for the first time, and adding water that is 10 times the total weight of the 5 traditional Chinese medicine for the second time, each extraction time is 3 hours, extraction temperature is 95° C., to filter to obtain a filtrate, and to concentrate the filtrate into a cream of 1.08/85° CCF.

Preferably, a detection method adopted in the step S3 is HPLC detection.

Preferably, a preparation process of the test sample described in the step S3 is: taking 0.3 g mixture of the cream and the fine powders, adding 200 mL of 75% formalin to dissolve, weighing, and then undergoing ultrasonic treatment for (15±5) minutes, after the ultrasonic treatment, adding 75% formalin to make up a mass loss, and then taking 2 mL of a dissolving solution and diluting to 10 mL with 75% formalin, passing through a 0.45 μm microporous membrane, and taking the filtrate as a solution to be tested.

Preferably, the HPLC detection conditions in the step S3 are: using Kromasil 100-5-C18 chromatographic column (250 mm×4.6 mm, 5 μm), with mobile phases using acetonitrile as an A phase and 0.1% phosphoric acid aqueous solution as a B phase, gradient eluting, with a flow rate being 1.0 mL·min−1, a detection wavelength being 254 nm, a column temperature being (30±0.5)° C., and an injection volume being 10 μL.

The present invention also claims for protection for use of the Fuke Qianjin Tablets in preparing drugs for treating a gynecological disease. Preferably, the gynecological disease is chronic pelvic inflammatory disease, chronic adnexitis or endometritis.

Specifically, Fuke Qianjin Tablets are made of Radix Et Caulis Flemingiae, Caulis Mahoniae, Herba Andrographis, Zanthoxylum dissitum Hemsl., Caulis Spatholobi, Radix Angelicae Sinensis, Radix Codonopsis, and Radix Rosa Laevigata as raw materials, each of the Fuke Qianjin Tablets contains not less than 0.008 mg of genistin, not less than 0.7 mg of Z-ligustilide, and a total amount of andrographolide and dehydroandrographolide is not less than 1.1 mg.

Preferably, each of the Fuke Qianjin Tablets contains not less than 0.012 mg of the genistin, not less than 0.8 mg of the Z-ligustilide, and the total amount of the andrographolide and the dehydroandrographolide is not less than 1.4 mg.

More preferably, each of the Fuke Qianjin Tablets contains 0.02 mg to 0.05 mg of the genistin, 1.15 mg to 1.65 mg of the Z-ligustilide, and the total amount of the andrographolide and the dehydroandrographolide is not less than 1.93 mg.

Fuke Qianjin Tablets are made of Radix Et Caulis Flemingiae, Caulis Mahoniae, Herba Andrographis, Zanthoxylum dissitum Hemsl., Caulis Spatholobi, Radix Angelicae Sinensis, Radix Codonopsis, and Radix Rosa Laevigata as raw materials, each of the Fuke Qianjin Tablets contains not less than 0.008 mg of genistin, not less than 0.015 mg of Z-3-butylidenephthalide, and a total amount of the andrographolide and the dehydroandrographolide is not less than 1.1 mg.

Preferably, each of the Fuke Qianjin Tablets contains not less than 0.012 mg of the genistin, not less than 0.02 mg of the Z-3-butylidenephthalide, and the total amount of the andrographolide and the dehydroandrographolide is not less than 1.4 mg.

More preferably, each of the Fuke Qianjin Tablets contains 0.02 mg to 0.05 mg of the genistin, 0.04 mg to 0.08 mg of the Z-3-butylidenephthalide, and the total amount of the andrographolide and the dehydroandrographolide is not less than 1.93 mg.

Fuke Qianjin Tablets are made of Radix Et Caulis Flemingiae, Caulis Mahoniae, Herba Andrographis, Zanthoxylum dissitum Hemsl., Caulis Spatholobi, Radix Angelicae Sinensis, Radix Codonopsis, and Radix Rosa Laevigata as raw materials, each of the Fuke Qianjin Tablets (according to the existing product and production index as a reference, the mass of each of the Fuke Qianjin Tablets is 0.32 g) contains not less than 0.008 mg of genistin, not less than 0.7 mg of Z-ligustilide, not less than 0.015 mg of Z-3-butylidenephthalide, and a total amount of andrographolide and dehydroandrographolide is not less than 1.1 mg.

Preferably, each of the Fuke Qianjin Tablets contains not less than 0.012 mg of the genistin, not less than 0.8 mg of the Z-ligustilide, not less than 0.02 mg of the Z-3-butylidenephthalide, and the total amount of the andrographolide and the dehydroandrographolide is not less than 1.4 mg.

More preferably, in each of the Fuke Qianjin Tablets, the total amount of the andrographolide and the dehydroandrographolide is not less than 1.93 mg.

More preferably, each of the Fuke Qianjin Tablets contains 0.02 mg to 0.05 mg of the genistin, 1.15 mg to 1.65 mg of the Z-ligustilide, 0.04 mg to 0.08 mg of the Z-3-butylidenephthalide, and the total amount of the andrographolide and the dehydroandrographolide is not less than 1.93 mg.

Preferably, the contents of the genistin, the Z-ligustilide and the Z-3-butylidenephthalide, and the total amount of the andrographolide and the dehydroandrographolide are determined by HPLC detection.

Preferably, a test sample for the HPLC detection is prepared by the following method: taking 10 Fuke Qianjin Tablets, removing coating, accurately weighing, porphyrizing, taking 0.3 g, placing in a container, accurately adding 20 mL of 75% formalin, ultrasonically extracting for (15±5) minutes, after cooling to room temperature, using 75% formalin to make up a mass loss, passing through a 0.45 μm microporous membrane, and taking the filtrate as a solution to be tested.

Preferably, the test sample for the HPLC detection is prepared by the following method: using Kromasil 100-5-C18 chromatographic column (250 mm×4.6 mm, 5 μm), with mobile phases using acetonitrile as an A phase and 0.1% phosphoric acid aqueous solution as a B phase, gradient eluting, with a flow rate being 1.0 mL·min−1, a detection wavelength being 254 nm, a column temperature being (30±0.5)° C., and an injection volume being 10 μL.

Preferably, the genistin is from the raw medicinal material Radix Et Caulis Flemingiae of the Fuke Qianjin Tablets, the Z-ligustilide and the Z-3-butylidenephthalide are from the raw medicinal material Radix Angelicae Sinensis of the Fuke Qianjin Tablet, and the andrographolide and the dehydroandrographolide are from the raw medicinal material Herba Andrographis of Fuke Qianjin Tablet.

Preferably, dosage of the Radix Angelicae Sinensis, the Radix Codonopsis, the Herba Andrographis and the Zanthoxylum dissitum Hemsl. is each 9% of a total amount of the medicinal materials; and dosage of the Radix Rosa Laevigata, the Caulis Spatholobi, the Caulis Mahoniae and the Radix Et Caulis Flemingiae is each 16% of the total amount of the medicinal materials.

Compared with the prior art, the present invention has the following beneficial effects.

The present invention has added a detection process in the preparation process of the product, and has established a new standard for controlling quality of the Fuke Qianjin Tablets through an analysis of chemical ingredients in the Fuke Qianjin Tablets. This standard adds a variety of core ingredient content to the existing pharmacopoeia standards. According to the Fuke Qianjin Tablets made in this range, the consistency of effects between different batches is more stable. Moreover, the more the types of core ingredients are limited, the more stable the consistency of the drug effect. Compared with the prior art, the Fuke Qianjin Tablets provided by the present invention has a better clinical treatment effect.

At the same time, the present invention adopts HPLC method for detection, which not only has high accuracy of detection results, but also rapid and simple detection process, which is convenient for the actual production process of the Fuke Qianjin Tablets of the present invention to simultaneously detect and monitor contents of multiple active ingredients in the original preparation process, which is conducive to the implementation of the new standard.

### DESCRIPTION OF EMBODIMENTS

The present invention is further described in detailed below in combination with specific embodiments, which are only used to explain the present invention, and are not used to limit the scope of the present invention. Unless otherwise specified, test methods used in the following embodiments are conventional methods. The materials and reagents used, unless otherwise specified, are commercially available reagents and materials.

### Embodiment 1 Fuke Qianjin Tablets

Formula of Fuke Qianjin Tablets: dosage of Radix Angelicae Sinensis, Radix Codonopsis, Herba Andrographis and Zanthoxylum dissitum Hemsl. was each 9% of a total amount of the medicinal materials; and dosage of Radix Rosa Laevigata, Caulis Spatholobi, Caulis Mahoniae and Radix Et Caulis Flemingiae was each 16% of the total amount of the medicinal materials. The total amount of the medicinal materials was 500 kg. The product was prepared by the following methods:

(1) Radix Angelicae Sinensis, Radix Codonopsis and Herba Andrographis were selected and crushed into fine powders of 100 meshes, and reserved for use at a powder yield of at least 93.3%;

(2) Zanthoxylum dissitum Hemsl. And Radix Rosa Laevigata, Caulis Spatholobi, Caulis Mahoniae and Radix Et Caulis Flemingiae were selected and added with water to extract twice, wherein water that was 10 times a total weight of the 5 traditional Chinese medicine was added for the first time, and water that was 10 times the total weight of the 5 traditional Chinese medicine was added for the second time, each extraction time was 3 hours, extraction temperature was 95° C., filtration was carried out to obtain a filtrate, and the filtrate was concentrated into a cream of 1.08/85° CCF; and

(3) the fine powders of (1) and the qualified cream of (2) were mixed for HPLC detection, by methods available in the field, a content of genistin was controlled not less than 0.00005 mg, a content of Z-ligustilide was controlled not less than 0.00394 mg and/or a content of Z-3-butylidenephthalide was controlled not less than 0.00008 mg, a content of andrographolide and dehydroandrographolide was controlled not less than 0.00619 mg; and then drying, tabletting and coating were carried out to obtain the Fuke Qianjin Tablets, and according to the existing product and production index as a reference, the mass of each Fuke Qianjin Tablet was 0.32 g.

When the detection result in the step (3) failed to meet the above conditions, and the contents of any one of the three active ingredients (genistin, Z-ligustilide, Z-3-butylidenephthalide) failed to meet the above conditions, a part of sample was reserved for use.

When the detection result in the step (3) failed to meet the above conditions, and the contents of the three active ingredients (genistin, Z-ligustilide, Z-3-butylidenephthalide) all failed to meet the above conditions, it was a comparative product and was reserved for use.

The Fuke Qianjin Tablets were prepared according to the above method, multiple batches of products were randomly selected from a long-term, large number of products, and HPLC detection was performed on each batch of the Fuke Qianjin Tablet products.

Preparation of a solution to be tested: 10 Fuke Qianjin Tablets of the same batch were randomly selected, with coating removed, accurately weighed, porphyrized, 0.3 g was taken and placed in a triangular flask with a ground stopper, 20 mL of 75% formalin was accurately added, weighing was performed, ultrasonic extraction was performed for 15 minutes at a power of 200 W and a frequency of 40 kHz, after cooled to room temperature, 75% formalin was used to make up a mass loss, after passing through a 0.45 μm microporous membrane, a filtrate was taken as the solution to be tested.

Kromasil 100-5-C18 chromatographic column (250 mm×4.6 mm, 5 μm) was used, mobile phases were acetonitrile (A phase) and 0.1% phosphoric acid aqueous solution (B phase), gradient elution was performed, with a flow rate of 1.0 mL·min−1, a detection wavelength of 254 nm, a column temperature of 30° C., and an injection volume of 10 μL.

### Embodiment 2

Fuke Qianjin Tablets, the steps (1) and (2) in the preparation process thereof are the same as in Embodiment 1, and the difference from Embodiment 1 is that, in the step S3, per milligram of the mixture, the content of the genistin was controlled not less than 0.00007 mg, the content of the Z-ligustilide was controlled not less than 0.0045 mg and/or the content of the Z-3-butylidenephthalide was controlled not less than 0.00011 mg, and the content of the andrographolide and the dehydroandrographolide was controlled not less than 0.00788 mg; and then drying, tabletting and coating were carried out to obtain the Fuke Qianjin Tablets, and according to the existing product and production index as a reference, the mass of each Fuke Qianjin Tablet was 0.32 g.

The Fuke Qianjin Tablets were prepared according to the above method, three batches of products were randomly selected from a long-term, large number of products, and HPLC detection was performed on each batch of the Fuke Qianjin Tablet products.

The HPLC detection results of the 6 batches randomly selected from the multiple batches of samples detected in Embodiment 1 and of the 6 batches randomly selected in the Embodiment 2 are shown in Table 1.

TABLE 1
Contents of five ingredients in Fuke Qianjin Tablets prepared in the Embodiments 1 and 2 (μg/tablet)

| | | | | | | Z-3- |
|---|---|---|---|---|---|---|
| | | Genistin/ | Andrographolide/ | Dehydroandrographolide/ | Z-ligustilide/ | butylidenephthalide/ |
| | Batch | μg | μg | μg | μg | μg |
| Embodiment 1 | Batch 1 | 12.03 ± 0.21 | 714.35 ± 4.36 | 556.55 ± 0.35 | 1122.15 ± 1.25 | 14.34 ± 0.35 |
| | Batch 2 | 16.11 ± 0.41 | 1089.17 ± 2.15 | 797.14 ± 4.74 | 1322.01 ± 1.3 | 12.14 ± 0.55 |
| | Batch 3 | 10.03 ± 0.23 | 1233.63 ± 10.36 | 622.02 ± 3.11 | 576.33 ± 8.14 | 22.14 ± 0.15 |
| | Batch 4 | 23.36 ± 0.55 | 1475.02 ± 8.16 | 890.14 ± 6.67 | 537.31 ± 1.44 | 33.02 ± 0.21 |
| | Batch 5 | 20.45 ± 0.23 | 655.15 ± 2.02 | 819.03 ± 2.55 | 1530.35 ± 11.21 | 42.36 ± 0.25 |
| | Batch 6 | 23.13 ± 0.44 | 1712.31 ± 6.24 | 830.12 ± 1.11 | 1241.25 ± 1.41 | 51.67 ± 0.74 |
| Embodiment 2 | Batch 1 | 16.67 ± 0.23 | 877.44 ± 6.25 | 702.31 ± 0.26 | 1663.33 ± 4.45 | 52.94 ± 0.71 |
| | Batch 2 | 34.51 ± 0.32 | 1057.81 ± 3.91 | 692.89 ± 0.75 | 775.75 ± 0.94 | 38.22 ± 0.13 |
| | Batch 3 | 23.63 ± 0.74 | 901.12 ± 1.82 | 838.15 ± 1.74 | 930.73 ± 2.43 | 46.53 ± 0.07 |
| | Batch 4 | 18.93 ± 0.28 | 1307.9 ± 5.66 | 740.75 ± 1.38 | 1381.3 ± 4.6 | 22.98 ± 0.59 |
| | Batch 5 | 41.07 ± 0.06 | 1055.12 ± 3.2 | 604.76 ± 0.78 | 1086.95 ± 3.93 | 38.68 ± 0.17 |
| | Batch 6 | 36.52 ± 0.11 | 1486.83 ± 4.02 | 919.73 ± 2.28 | 1281.85 ± 3.67 | 48.91 ± 0.55 |

### Comparative Example 1 Fuke Qianjin Tablets (Prepared using an Existing Method)

The mixed cream in which the contents of genistin, Z-ligustilide and Z-3-butylidenephthalide in the mixed cream in the step (3) of Embodiment 1 were not within the range required by Embodiment 1 was selected and directly performed with drying, tabletting and coating to obtain the Fuke Qianjin Tablets. According to the 2015 edition of the Pharmacopoeia of the People's Republic of China, the mass of each Fuke Qianjin Tablet is 0.32 g. The same method was used for HPLC detection for each batch of the Fuke Qianjin Tablets.

The detection conditions and methods are the same as Embodiment 1, and the detection results are shown in Table 2.

TABLE 2
Contents of five ingredients in Fuke Qianjin Tablets
prepared in Comparative Example 1 (μg/tablet)

| | Batch 1 | Batch 2 | Batch 3 | Batch 4 |
|---|---|---|---|---|
| Genistin/mg | 7.73 ± 0.11 | 6.82 ± 0.31 | 7.75 ± 0.25 | 5.62 ± 0.15 |
| Andrographolide/mg | 556.78 ± 3.24 | 610.22 ± 2.12 | 494.21 ± 0.98 | 565.12 ± 2.43 |
| Dehydroandrographolide/mg | 431.13 ± 2.14 | 452.45 ± 1.05 | 475.33 ± 5.41 | 395.13 ± 2.22 |
| Z-ligustilide/mg | 644.42 ± 2.51 | 590.13 ± 0.24 | 538.32 ± 3.31 | 608.17 ± 7.01 |
| Z-3-butylidenephthalide/mg | 11.49 ± 0.17 | 13.36 ± 0.37 | 14.47 ± 0.12 | 11.14 ± 0.32 |

It can be seen from Table 2 that in the Fuke Qianjin Tablets prepared according to the existing method, except that the content of andrographolide and dehydroandrographolide meets the requirements of the Pharmacopoeia, the contents of other active ingredients vary greatly between batches, which can easily lead to instability of drug efficacy.

### Embodiment 3 In Vitro Efficacy Test

Medicines or materials used: croton oil, provided by Nanjing Institute of Dermatology; carrageenan, produced by Wako Pure Chemical Industries, Ltd.; nutrient broth medium, product of Guangdong Huankai Microbial Technology Co., Ltd.; and mould medium, provided by China National Institute for the Control of Pharmaceutical and Biological Products.

The Fuke Qianjin Tablets prepared by Embodiment 1 (batch 1 and batch 6) and Comparative Example 1 (batch 1) were used as samples. Kunming mice of clean grade and SD rats used were provided by the Hunan Institute for Drug Control; Escherichia coli ATCC25922, Staphylococcus aureus ATCC25923, beta hemolytic Streptococcus ATCC32172 were all provided by the Provincial Health and Epidemic Prevention Station, Candida albicans, isolated from the clinic, was provided by the Bacteria Room of the Clinical Laboratory Department of the Third Affiliated Hospital of Hunan Medical University.

### 1) In Vitro Antibacterial Test on Standard Bacteria

The results are shown in Table 3 to Table 5.

TABLE 3
Antibacterial test results of Fuke Qianjin Tablets of
batch 1 in Embodiment 1 (liquid test tube method)

| | Bacterial | | | | | | |
|---|---|---|---|---|---|---|---|
| | concentration | Drug concentration (%) | | | | | Blank |
| Bacteria | (GFu/mL) | 50.0 | 25.0 | 12.5 | 6.25 | 3.13 | control |
| Escherichia coli | 10⁶ | == | == | == | +++ | +++ | +++ |
| Staphylococcus aureus | 10⁶ | == | == | == | == | ++ | +++ |
| Beta hemolytic streptococcus | 10⁶ | == | == | == | +++ | +++ | +++ |
| Candida albicans | 10⁶ | == | == | == | ++ | +++ | +++ |

TABLE 4
Antibacterial test results of Fuke Qianjin Tablets of
batch 6 in Embodiment 1 (liquid test tube method)

| | Bacterial | | | | | | |
|---|---|---|---|---|---|---|---|
| | concentration | Drug concentration (%) | | | | | Blank |
| Bacteria | (GFu/mL) | 50.0 | 25.0 | 12.5 | 6.25 | 3.13 | control |
| Escherichia coli | 10⁶ | == | == | == | +++ | +++ | +++ |
| Staphylococcus aureus | 10⁶ | == | == | == | == | ++ | +++ |
| Beta hemolytic streptococcus | 10⁶ | == | == | == | +++ | +++ | +++ |
| Candida albicans | 10⁶ | == | == | == | ++ | +++ | +++ |

TABLE 5
Antibacterial test results of Fuke Qianjin Tablets of batch
1 in Comparative Example 1 (liquid test tube method)

| | Bacterial | | | | | | |
|---|---|---|---|---|---|---|---|
| | concentration | Drug concentration (%) | | | | | Blank |
| Bacteria | (GFu/mL) | 50.0 | 25.0 | 12.5 | 6.25 | 3.13 | control |
| Escherichia coli | 10⁶ | == | == | == | +++ | +++ | +++ |
| Staphylococcus aureus | 10⁶ | == | == | == | == | ++ | +++ |
| Beta hemolytic streptococcus | 10⁶ | == | == | == | +++ | +++ | +++ |
| Candida albicans | 10⁶ | == | == | == | ++ | +++ | +++ |

== indicates that there is no bacterial growth in two repeated experiments, and +, ++, +++ indicate the degree of cell growth, respectively

The experimental results of Table 3 to Table 5 show that: the Fuke Qianjin Tablets of Embodiment 1 and Comparative Example 1 have minimum inhibitory concentrations of 12.5, 6.25, 12.5, 12.5 for Escherichia coli, Staphylococcus aureus, beta hemolytic Streptococcus and Candida albicans, respectively, and the inhibitory concentrations of the two are the same.

### 2) In Vitro Antibacterial Experiment on Clinically Isolated Pathogenic Bacteria

Escherichia coli, Staphylococcus aureus, beta hemolytic Streptococcus, and Candida albicans were all isolated from clinical patients and identified by bacteriology, provided by the Bacteria Room of the Clinical Laboratory Department of the Third Affiliated Hospital of Hunan Medical University.

The results are shown in Table 6 to Table 8.

TABLE 6
Antibacterial test results of Fuke Qianjin Tablets of batch 1
of Embodiment 1 on clinical isolation (liquid test tube method)

| | Plant | Bacterial | |
|---|---|---|---|
| | number | concentration | Minimal inhibitory |
| Bacterial strain | (plant) | (GFu/mL) | concentration (%) |
| Escherichia coli | 32 | 10⁵~10⁶ | 50.0~25.0 |
| Staphylococcus aureus | 23 | 10⁵~10⁶ | 12.5~3.13 |
| Beta hemolytic | 20 | 10⁵~10⁶ | 50.0~25.0 |
| streptococcus | | | |
| Candida albicans | 27 | 10⁵~10⁶ | 12.5~3.13 |

TABLE 7
Antibacterial test results of Fuke Qianjin Tablets of batch 6
of Embodiment 1 on clinical isolation (liquid test tube method)

| | Plant | Bacterial | Minimal |
|---|---|---|---|
| | number | concentration | inhibitory |
| Bacterial strain | (plant) | (GFu/mL) | concentration (%) |
| Escherichia coli | 32 | 10⁵~10⁶ | 50.0~25.0 |
| Staphylococcus aureus | 23 | 10⁵~10⁶ | 12.5~3.13 |
| Beta hemolytic | 20 | 10⁵~10⁶ | 50.0~25.0 |
| streptococcus | | | |
| Candida albicans | 27 | 10⁵~10⁶ | 12.5~3.13 |

TABLE 8
Antibacterial test results of Fuke Qianjin Tablets
of batch 1 of Comparative Example 1 on clinical
isolation (liquid test tube method)

| | Plant | Bacterial | |
|---|---|---|---|
| | number | concentration | Minimal inhibitory |
| Bacterial strain | (plant) | (GFu/mL) | concentration (%) |
| Escherichia coli | 32 | 10⁵~10⁶ | 50.0~25.0 |
| Staphylococcus aureus | 23 | 10⁵~10⁶ | 12.5~3.13 |
| Beta hemolytic | 20 | 10⁵~10⁶ | 50.0~25.0 |
| streptococcus | | | |
| Candida albicans | 27 | 10⁵~10⁶ | 12.5~3.13 |

The experimental results of Table 6 to Table 8 show that the Fuke Qianjin Tablets prepared by Embodiment 1 and Comparative Example 1 have the same inhibitory effect on the four clinically isolated bacteria, and the effect is consistent.

### 3) Antibacterial Experiment In Vivo

Protective effect on Escherichia coli-infected mice: 150 Kunming mice, both male and female, weighing 20-22 g, were divided into 15 groups (n=10), five groups were used for the Qianjin Tablets of batch 1 of Embodiment 1, five groups were used for the Qianjin Tablets of batch 6 of Embodiment 1, and five groups were used for the Qianjin Tablets of batch 1 of Comparative Example 1, and the doses were 17.3, 24.7, 35.3, 50.4 and 72.0 g crude drug/kg.

Volume of intragastric administration was 0.5 mL/20 g, and each mouse in each group was injected with 0.5 mL of Escherichia coli (106 FGu/mL) decoction culture solution one hour after the administration. At 12 hours and 24 hours after the mice were infected with the bacteria, the mice were administered twice, and then observed for seven days, and the number of animal deaths was recorded. The peak of animal deaths was between 24 hours and 48 hours.

TABLE 9
Protective effect of Fuke Qianjin Tablets
on Escherichia coli-infected mice

| | Dosage | Animal | | Protection |
|---|---|---|---|---|
| Group | (g/kg) | numbers | Death count | rate (%) |
| Fuke Qianjin Capsule | 72.0 | 10 | 4 | 60 |
| (batch 1 of | 50.4 | 10 | 6 | 40 |
| Embodiment) | 35.3 | 10 | 8 | 20 |
| | 24.7 | 10 | 9 | 10 |
| | 17.3 | 10 | 0 | 0 |
| Fuke Qianjin Capsule | 72.0 | 10 | 4 | 60 |
| (batch 6 of | 50.4 | 10 | 6 | 40 |
| Embodiment) | 35.3 | 10 | 8 | 20 |
| | 24.7 | 10 | 9 | 10 |
| | 17.3 | 10 | 0 | 0 |
| Fuke Qianjin Capsule | 72.0 | 10 | 4 | 60 |
| (batch 1 of | 50.4 | 10 | 6 | 40 |
| Comparative | 35.3 | 10 | 8 | 20 |
| Example 1) | 24.7 | 10 | 9 | 10 |
| | 17.3 | 10 | 0 | 0 |

The experimental results in Table 9 show that the Fuke Qianjin Tablets prepared by Embodiment 1 and Comparative Example 1 have a certain protective effect on Escherichia coli-infected mice in the high-dose group.

### 4) Anti-Inflammatory Effect (Influence on Croton Oil-Induced Swelling in Mice)

Preparation of drugs to be used: the ready-to-use Fuke Qianjin Tablets were taken and prepared into suspensions of different concentrations with distilled water for mice. Intragastric administration was performed once a day, and the liquid for intragastric administration was used it right after it was prepared.

100 mice, male, weighing 24-26 g, were equally divided into 10 groups (n=10), and were given different concentrations of liquid medicine by intragastric administration, and the control group was given with equal volume of distilled water by intragastric administration once a day for 7 days, 1 hour after the last administration was performed, the right ear of each mouse was applied with 0.1 mL of 2% croton oil (2% croton oil, 20% anhydrous ethanol, 5% distilled water and 73% diethyl ether), and no treatment was applied on the left ear of all mice. On the 7th day, 4 hours after the last administration, the mice were sacrificed by cervical dislocation, the ears were cut off, punched and weighed, and the swelling degree of each group of mice was calculated.

TABLE 10
Influence of Fuke Qianjin Tablets on croton oil-induced
ear swelling in mice (X ± SD, n = 10)

| | Dosage | Weight of | Weight of | Swelling | Inhibition |
|---|---|---|---|---|---|
| Group | (g/kg) | right ear (mg) | left ear (mg) | degree (mg) | rate (%) |
| Control group | 16 g/kg | 30.3 ± 3.7 | 9.5 ± 1.0 | 20.8 ± 4.1 | — |
| | distilled | | | | |
| | water | | | | |
| Fuke Qianjin | 16 | 19.5 ± 1.7 | 9.5 ± 1.2 | 10.0 ± 0.8*** | 51.9% |
| Tablets | 8 | 20.8 ± 2.4 | 9.2 ± 1.1 | 11.6 ± 0.4*** | 44.2% |
| (batch 6 of | 4 | 22.5 ± 1.1 | 9.1 ± 1.2 | 13.4 ± 0.6** | 35.6% |
| Embodiment 1) | | | | | |
| Fuke Qianjin | 16 | 22.8 ± 2.1 | 9.3 ± 1.1 | 13.5 ± 0.5** | 35.1% |
| Tablets | 8 | 24.0 ± 4.5 | 9.3 ± 1.0 | 14.7 ± 0.7** | 29.3% |
| (batch 1 of | 4 | 24.2 ± 1.1 | 9.1 ± 1.0 | 15.1 ± 0.8* | 27.4% |
| Embodiment 1) | | | | | |
| Fuke Qianjin | 16 | 23.8 ± 2.1 | 9.7 ± 1.0 | 14.1 ± 0.7** | 32.2% |
| Tablets | 8 | 24.3 ± 1.7 | 9.4 ± 1.2 | 14.9 ± 0.7** | 28.4% |
| (batch 1 of | 4 | 24.1 ± 2.3 | 8.9 ± 1.4 | 15.2 ± 0.6* | 26.9% |
| Comparative | | | | | |
| Example 1) | | | | | |

Compared with the control group,
*P > 0.05,
**P < 0.05,
***P < 0.01

The results in Table 10 show that: compared with the control group, the Fuke Qianjin Tablets prepared by Embodiment 1 and Comparative Example 1 can both significantly inhibit croton oil-induced ear swelling in mice; however, the inhibition effect of the two batches in Embodiment 1 is significantly better than the Fuke Qianjin Tablets of Comparative Example 1.

### 5) Influence on Carrageenan-Induced Footpad Swelling in Rats

70 SD rats, males, were divided into seven groups (n=10), and they were given different concentrations of liquid medicine by intragastric administration, and the control group was given with the same volume of distilled water by intragastric administration, once a day for seven days, 1 hour after the last administration was performed, 0.1 mL of carrageenan was injected into the bottom of the right pedal of each rat to cause inflammation. 2 hours after the inflammation, it is administered again. In addition to measuring the size of the normal pedal before the inflammation, the size of pedal was measured every 1 hour after the inflammation for a total of 6 times, and the swelling degree was calculated.

TABLE 11
Influence of Fuke Qianjin Tablets on carrageenan-induced footpad swelling in rats (X ± cm, n = 10)

| | | Pedal | | | | | | |
|---|---|---|---|---|---|---|---|---|
| | | size | | | | | | |
| | | before | | | | | | |
| | Dosage | experiment | Swelling degree after inflammation (cm) | | | | | |
| Group | (g/kg) | (cm) | 1 hour | 2 hours | 3 hours | 4 hours | 5 hours | 6 hours |
| Control | 10.08 g/kg | 2.4 ± 0.3 | 0.4 ± 0.1 | 0.68 ± 0.1 | 0.9 ± 0.1 | 0.73 ± 0.3 | 0.55 ± 0.1 | 0.43 ± 0.2 |
| group | distilled | | | | | | | |
| | water | | | | | | | |
| Fuke Qianjin | 10.08 | 2.4 ± 0.1 | 0.26 ± 0.1** | 0.35 ± 0.2** | 0.52 ± 0.1* | 0.48 ± 0.3** | 0.4 ± 0.1** | 0.26 ± 0.1*** |
| Tablets | 5.04 | 2.5 ± 0.1 | 0.32 ± 0.1* | 0.40 ± 0.2* | 0.58 ± 0.1** | 0.56 ± 0.3** | 0.41 ± 0.1** | 0.30 ± 0.1** |
| (batch 6 of | 2.52 | 2.7 ± 0.2 | 0.35 ± 0.1 | 0.45 ± 0.2* | 0.66 ± 0.1* | 0.60 ± 0.3* | 0.46 ± 0.1* | 0.33 ± 0.1** |
| Embodiment 1) | | | | | | | | |
| Fuke Qianjin | 10.08 | 2.4 ± 0.3 | 0.32 ± 0.1* | 0.4 ± 0.2* | 0.7 ± 0.1* | 0.6 ± 0.3* | 0.4 ± 0.1* | 0.3 ± 0.1* |
| Tablets | 5.04 | 2.7 ± 0.1 | 0.34 ± 0.1* | 0.43 ± 0.2* | 0.71 ± 0.1* | 0.62 ± 0.3* | 0.41 ± 0.1* | 0.35 ± 0.1 |
| (batch 1 of | 2.52 | 2.9 ± 0.3 | 0.40 ± 0.1 | 0.49 ± 0.2 | 0.78 ± 0.1 | 0.67 ± 0.3 | 0.46 ± 0.1 | 0.36 ± 0.1 |
| Embodiment 1) | | | | | | | | |
| Fuke Qianjin | 10.08 | 2.4 ± 0.3 | 0.33 ± 0.1* | 0.4 ± 0.2* | 0.7 ± 0.1* | 0.6 ± 0.3* | 0.41 ± 0.1* | 0.3 ± 0.1* |
| Tablets | 5.04 | 2.6 ± .3 | 0.37 ± 0.1 | 0.47 ± 0.2* | 0.72 ± 0.1* | 0.64 ± 0.3 | 0.41 ± 0.1* | 0.39 ± 0.1 |
| (batch 1 of | 2.52 | 2.9 ± 0.3 | 0.41 ± 0.1 | 0.52 ± 0.2 | 0.79 ± 0.1 | 0.69 ± 0.3 | 0.47 ± 0.1 | 0.38 ± 0.1 |
| Comparative | | | | | | | | |
| Example 1) | | | | | | | | |

Compared with the control group,
*P > 0.05,
**P < 0.05,
***P < 0.01

The results in Table 11 show that: compared with the control group, the Fuke Qianjin Tablets prepared by Embodiment 1 and Comparative Example 1 can significantly inhibit the carrageenan-induced footpad swelling in rats; however, the two batches of the Fuke Qianjin Tablets in Embodiment 1 have better inhibitory effects on the carrageenan-induced footpad swelling in rats than the Fuke Qianjin Tablets of Comparative Example 1, especially in the 3-4 hours period after the administration.

### 6) Influence on Induced Painful Mice Induced by Acetic Acid

100 mice, half male and half male, weighing 20-22 g, were randomly divided into 10 groups (n=10), and they were given different concentrations of liquid medicine by intragastric administration, and the control group was given with the same volume of distilled water by intragastric administration, one hour after the administration, each mouse was injected with 0.2 mL of 0.6% acetic acid. 5 minutes after the injection, the recording was started, and the number of mouse writhing times in 10 minutes was recorded.

TABLE 12
Influence of Fuke Qianjin Tablets on painful mice induced
by acetic acid (X ± SD, n = 10)

| | Dosage | | Inhibition |
|---|---|---|---|
| Group | (g/kg) | Writhing times (times) | rate (%) |
| Control group | 18.2 g/kg | 22.4 ± 1.8 | — |
| | distilled | | |
| | water | | |
| Fuke Qianjin | 18.2 | 8.6 ± 1.1*** | 61.6% |
| Tablets | 9.1 | 10.1 ± 1.5*** | 54.9% |
| (batch 6 of | 3.6 | 12.6 ± 1.2*** | 43.75% |
| Embodiment 1) | | | |
| Fuke Qianjin | 18.2 | 9.9 ± 1.2*** | 55.8% |
| Tablets | 9.1 | 11.3 ± 1.3*** | 49.5% |
| (batch 1 of | 3.6 | 13.6 ± 1.7*** | 39.2% |
| Embodiment 1) | | | |
| Fuke Qianjin | 18.2 | 10.1 ± 1.8*** | 54.9% |
| Tablets | 9.1 | 11.6 ± 1.6*** | 48.2% |
| (batch 1 of | 3.6 | 14.1 ± 1.5*** | 37.1% |
| Comparative | | | |
| Example 1) | | | |

Compared with the control group,
***P < 0.01

The results in Table 12 show that: compared with the control group, the Fuke Qianjin Tablets prepared by Embodiment 1 and Comparative Example 1 can significantly reduce the number of writhing times of painful mice induced by acetic acid; however, the effect of the two batches of the Fuke Qianjin Tablets in Embodiment 1 on the pain induced by acetic acid is significantly better than the Fuke Qianjin Tablets of Comparative Example 1.

### 7) Influence on Pain Threshold of Painful Mice Induced by Hot Plate Test

100 female mice with a pain threshold of less than 30 seconds, weighing 20-22 g, were divided into 10 groups (n=10), and they were given by one-time intragastric administration according to the dose of painful mice induced by acetic acid, and according to the method in the literature “Analgesic activity of met-enkephalin modified by polyethylene glycol through intravenous injection to the painful mice induced by hot plate”, the pain thresholds were measured at 55±0.5° C. before administration and 30, 60 and 90 minutes after administration, and the results are shown in Table 16.

TABLE 13
Influence of Fuke Qianjin Tablets on pain threshold of painful mice induced by hot plate test (X ± SD, n = 10)

| | | | 30 minutes | | 60 minutes | | 90 minutes | |
|---|---|---|---|---|---|---|---|---|
| | | Pain | Pain | Percent | Pain | | Pain | Percent |
| | | threshold | threshold | age | threshold | Percentage | threshold | age |
| | | before | after | increase | after | increase | after | increase |
| | Dosage | administration | administration | in pain | administration | in pain | administration | in pain |
| Group | (g/kg) | (s) | (s) | threshold/% | (s) | threshold/% | (s) | threshold/% |
| Control | 18.2 g/kg | 18.8 ± 6.5 | 24.8 ± 5.5 | 31.9 | 28.8 ± 4.5 | 53.2 | 27.3 ± 7.5 | 45.2 |
| group | water | | | | | | | |
| Fuke Qianjin | 18.2 | 18.1 ± 5.5 | 55.1 ± 6.3*** | 204.4 | 51.3 ± 7.2*** | 183.4 | 48.8 ± 3.5*** | 169.6 |
| Tablets | 9.1 | 18.7 ± 6.1 | 51.5 ± 6.1*** | 175.4 | 48.2 ± 3.5*** | 157.8 | 46.8 ± 4.5*** | 150.3 |
| (batch 6 of | 3.6 | 18.0 ± 3.5 | 46 ± 5.5*** | 155.6 | 43.2 ± 4.3** | 140 | 40.8 ± 4.8** | 126.7 |
| Embodiment 1) | | | | | | | | |
| Fuke Qianjin | 18.2 | 18.1 ± 2.5 | 52 ± 5.1*** | 187.3 | 48.3 ± 5.7*** | 166.9 | 45.6 ± 6.5** | 151.9 |
| Tablets | 9.1 | 18.2 ± 6.1 | 48 ± 6.1*** | 163.7 | 45 ± 6.2** | 147.3 | 42 ± 4.7** | 130.8 |
| (batch 1 of | 3.6 | 18.3 ± 5.1 | 43.2 ± 4.5** | 136.1 | 42.5 ± 5.2** | 132.2 | 40.3 ± 5.5** | 120.2 |
| Embodiment 1) | | | | | | | | |
| Fuke Qianjin | 18.2 | 18.4 ± 6.2 | 51.1 ± 5.3*** | 177.7 | 48.5 ± 5.2*** | 163.6 | 44.2 ± 4.5** | 140.2 |
| Tablets | 9.1 | 18.9 ± 6.4 | 49.3 ± 5.1*** | 160.8 | 45.1 ± 4.5** | 138.6 | 42.8 ± 3.5** | 126.5 |
| (batch 1 of | 3.6 | 18.0 ± 6.5 | 41.2 ± 4.5** | 128.9 | 40 ± 5.3** | 122.2 | 37.8 ± 3.8** | 110.0 |
| Comparative | | | | | | | | |
| Example 1) | | | | | | | | |

Compared with the control group,
**P < 0.05,
***P < 0.01

The results in Table 13 show that compared with the control group, the Fuke Qianjin Tablets of Embodiment 1 and Comparative Example 1 can significantly increase the pain threshold of painful mice induced by hot plate excitement; however, the percentage increase in pain threshold of painful mice induced by hot plate excitement of the two batches of Fuke Qianjin Tablets in Embodiment 1 is significantly higher than the Fuke Qianjin Tablets of Comparative Example 1.

### 8) Influence on Hemorrhagic Blood Deficiency Mice

110 mice, both male and female, weighing 20-22 g, were divided into 11 groups (n=10), First, blood was taken to measure normal values RBC and HB of the mice, then, except for the normal control group, each mouse in the other groups was bled 0.5 mL from the orbital venous plexus, 24 hours later, blood was taken to measure the RBC and HB values of the mice, and then different doses of medicine were given by intragastric administration, once a day for seven days, 24 hours after the last administration, blood was taken from the orbital venous plexus of the mice to measure the RBC and HB values, and the results are shown in Table 14.

TABLE 14
Influence of Fuke Qianjin Tablets on hemorrhagic blood deficiency mice (X ± SD, n = 10)

| | | RBC(×1012/L) | | | HB(g/L) | | |
|---|---|---|---|---|---|---|---|
| | | | After | | | After | |
| | Dosage | Before | blood | After | Before | blood | After |
| Group | (g/kg) | blood loss | loss | treatment | blood loss | loss | treatment |
| Normal | Equal | 6.87 ± 0.62 | 6.20 ± 0.51 | 6.54 ± 0.43 | 137.0 ± 4.6 | 124.0 ± 2.9 | 129.0 ± 3.8 |
| control group | volume | | | | | | |
| | of water | | | | | | |
| Model | Equal | 6.88 ± 0.41 | 3.55 ± 0.62 | 5.01 ± 0.33 | 134.0 ± 6.6 | 77.8 ± 5.3 | 98.0 ± 7.6 |
| control group | volume | | | | | | |
| | of water | | | | | | |
| Fuke Qianjin | 18.2 | 6.77 ± 0.36 | 3.70 ± 0.16 | 5.68 ± 0.22** | 131.0 ± 5.2 | 76.9 ± 3.2 | 109.0 ± 4.6** |
| Tablets | 9.1 | 7.01 ± 0.44 | 3.60 ± 0.26 | 5.44 ± 0.27* | 132.0 ± 5.5 | 73.9 ± 1.2 | 101.0 ± 2.6* |
| (batch 6 of | 3.6 | 7.03 ± 0.55 | 3.56 ± 0.09 | 5.11 ± 0.27 | 137.0 ± 3.8 | 74.3 ± 1.3 | 97.0 ± 2.4 |
| Embodiment 1) | | | | | | | |
| Fuke Qianjin | 18.2 | 7.04 ± 0.24 | 3.99 ± 0.26 | 5.68 ± 0.55* | 134.0 ± 3.2 | 78.9 ± 1.2 | 107.0 ± 2.6* |
| Tablets | 9.1 | 7.02 ± 0.6 | 3.74 ± 0.12 | 5.32 ± 0.24 | 129.0 ± 2.2 | 78.9 ± 1.2 | 105.0 ± 4.6* |
| (batch 1 of | 3.6 | 7.05 ± 2.6 | 3.84 ± 0.16 | 5.31 ± 0.22 | 139.0 ± 5.2 | 76.1 ± 3.2 | 101.0 ± 4.6 |
| Embodiment 1) | | | | | | | |
| Fuke Qianjin | 18.2 | 7.01 ± 0.16 | 3.80 ± 0.28 | 5.52 ± 0.27* | 134.0 ± 2.2 | 77.9 ± 3.1 | 106.0 ± 1.6* |
| Tablets | 9.1 | 7.04 ± 0.26 | 3.50 ± 0.64 | 5.15 ± 0.32 | 131.0 ± 3.2 | 78.9 ± 2.2 | 103.0 ± 1.6* |
| (batch 1 of | 3.6 | 7.01 ± 0.61 | 3.72 ± 0.16 | 5.29 ± 0.28 | 131.8 ± 5.2 | 76.9 ± 3.2 | 97.0 ± 4.6 |
| Comparative | | | | | | | |
| Example 1) | | | | | | | |

Compared with the model group,
*P > 0.05,
**P < 0.05

The results in Table 14 show that, compared with the control group, the Fuke Qianjin Tablets of Embodiment 1 and Comparative Example 1 both have a significant blood-enriching effect on hemorrhagic blood deficiency mice; however, the two batches of Fuke Qianjin Tablets in Embodiment 1 have a significantly higher blood-enriching effect on hemorrhagic blood deficiency mice than the Fuke Qianjin Tablets of Comparative Example.

Throughout the above experimental results, it can be seen that the Fuke Qianjin Tablets of Embodiment 1 and Comparative Example 1 have the same in vitro minimal inhibitory concentration for Escherichia coli, Staphylococcus aureus, beta hemolytic Streptococcus and Candida albicans, as well as the minimal inhibitory concentration of the above four clinically isolated bacteria.

However, in mice in vivo experiments, the Fuke Qianjin Tablets of the two batches in Embodiment 1 and in Comparative Example 1 can significantly inhibit croton oil-induced ear swelling in mice and carrageenan-induced footpad swelling in rats; reduce the number of writhing times in mice induced by acetic acid, and increase the pain threshold of painful mice induced by hot plate test; and can also have a significant blood-enriching effect on hemorrhagic blood deficiency mice; however, The above-mentioned effects of the Fuke Qianjin Tablets prepared in the two batches in Embodiment 1 have a certain degree of improvement compared with the Fuke Qianjin Tablets of Comparative Example 1, and the effects are better than the Fuke Qianjin Tablets of Comparative Example 1.

### Embodiment 4 Clinical Results

In order to compare whether there is a difference between the Fuke Qianjin Tablets that the present invention controls the content of multiple active ingredients and the Fuke Qianjin Tablets prepared by the original method (i.e., Comparative Example 1), a clinical trial was conducted. In accordance with the requirements of relevant new drug research, each test site has formulated clinical research principles, established diagnostic criteria, inclusion criteria, and rejection criteria, and used this as a guideline to collect observation cases. At the same time, method of taking medicine was followed in the instructions attached to the medicine during use, the medicine was started taking when seeing a doctor, 7 days as a course of treatment, two consecutive courses of treatment, and clinical symptoms and changes in symptoms were collected according to the planned clinical observation form.

Table 15 shows the clinical changes of 240 patients with chronic pelvic inflammatory disease in the Second Affiliated Hospital of Hunan University of Traditional Chinese Medicine, Hunan Academy of Traditional Chinese Medicine, and the First Affiliated Hospital of Hunan Medical University after taking qianjin tablets for two courses, divided into 3 groups, 80 patients in each group. The specific results are shown in Table 15.

TABLE 15
Patients with chronic pelvic inflammatory disease getter better after treatment and percentage table

| | | | | | Whole | | | | | |
|---|---|---|---|---|---|---|---|---|---|---|
| | Symptom | Low | | Lack of | body | | | Soreness of | Irregular | Increased |
| | name | fever | Fatigue | energy | discomfort | Insomnia | Hypogastralgia | waist | menstruation | leucorrhea |
| Qianjin | Number of | 10 | 38 | 30 | 25 | 16 | 58 | 47 | 20 | 46 |
| tablets | people | | | | | | | | | |
| (batch 6 of | before | | | | | | | | | |
| Embodiment 1) | treatment | | | | | | | | | |
| | Number of | 10 | 32 | 28 | 21 | 12 | 54 | 41 | 18 | 45 |
| | people | | | | | | | | | |
| | getting | | | | | | | | | |
| | better | | | | | | | | | |
| | Rate of | 100 | 84.2 | 93.3 | 84.0 | 75 | 93.1 | 87.2 | 90 | 97.8 |
| | getting | | | | | | | | | |
| | better % | | | | | | | | | |
| Qianjin | Number of | 9 | 36 | 31 | 27 | 15 | 55 | 47 | 22 | 48 |
| tablets | people | | | | | | | | | |
| (batch 1 of | before | | | | | | | | | |
| Embodiment 1) | treatment | | | | | | | | | |
| | Number of | 9 | 30 | 26 | 22 | 10 | 49 | 36 | 18 | 42 |
| | people | | | | | | | | | |
| | getting | | | | | | | | | |
| | better | | | | | | | | | |
| | Rate of | 100 | 83.3 | 83.9 | 81.5 | 66.7 | 89 | 76.6 | 81.2 | 91.7 |
| | getting | | | | | | | | | |
| | better % | | | | | | | | | |
| Qianjin | Number of | 10 | 37 | 31 | 26 | 15 | 56 | 48 | 21 | 49 |
| tablets | people | | | | | | | | | |
| (batch 1 of | before | | | | | | | | | |
| Comparative | treatment | | | | | | | | | |
| Example 1) | Number of | 9 | 30 | 25 | 21 | 10 | 49 | 36 | 17 | 43 |
| | people | | | | | | | | | |
| | getting | | | | | | | | | |
| | better | | | | | | | | | |
| | Rate of | 90 | 81.1 | 80.1 | 80.1 | 66.7 | 87.5 | 75 | 81.0 | 90.0 |
| | getting | | | | | | | | | |
| | better % | | | | | | | | | |

The effect statistics of 240 patients with chronic pelvic inflammatory disease taking Fuke Qianjin Tablets were listed. Tables 16 to 18 are the examination results of 240 patients with adnexitis, wherein Table 16 is the examination status, Table 17 is the efficacy statistics, and Table 18 is the change in symptoms before and after taking the Fuke Qianjin Tablets.

TABLE 16
Examination of patients with adnexitis

| | Number | Obvious | General | Light |
|---|---|---|---|---|
| Site | of cases | tenderness | tenderness | tenderness |
| Unilateral adnexitis | 102 | 35 | 52 | 15 |
| Bilateral adnexitis | 138 | 60 | 62 | 16 |
| Total | 240 | 95 | 114 | 31 |

TABLE 17
Efficacy of Qianjin Tablets in patients with annexitis

| | Bilateral adnexitis | | | Unilateral adnexitis | | | Overal efficacy | | |
|---|---|---|---|---|---|---|---|---|---|
| Site | Markedly | Getting | | Markedly | Getting | | Markedly | Getting | |
| Efficacy | effective | better | Noneffective | effective | better | Noneffective | effective | better | Noneffective |
| Number of cases | 23 | 21 | 2 | 21 | 12 | 1 | 44 | 33 | 3 |
| (batch 6 of | | | | | | | | | |
| Embodiment 1) | | | | | | | | | |
| Number of cases | 20 | 23 | 3 | 18 | 14 | 2 | 38 | 37 | 5 |
| (batch 1 of | | | | | | | | | |
| Embodiment 1) | | | | | | | | | |
| Number of cases | 20 | 22 | 4 | 18 | 14 | 2 | 38 | 36 | 6 |
| (batch 1 of | | | | | | | | | |
| Comparative | | | | | | | | | |
| Example) | | | | | | | | | |

TABLE 18
Changes in symptoms of patients with adnexitis before and after taking Fuke Qianjin Tablets

| | | Soreness | | | | |
|---|---|---|---|---|---|---|
| | | of waist | | | | |
| | | and | | | Increased | |
| | | tenesmus | hypogastralgia | Lumbago | leucorrhea | dysmenorrhea |
| Fuke Qianjin | Number of | 60 | 52 | 53 | 41 | 45 |
| Tablet | people | | | | | |
| (batch 6 of | before | | | | | |
| Embodiment 1) | treatment | | | | | |
| | Significantly | 51 | 49 | 51 | 40 | 36 |
| | reduced | | | | | |
| | Getting | 8 | 3 | 2 | 1 | 6 |
| | better | | | | | |
| | Noneffective | 1 | 0 | 0 | 0 | 3 |
| | Rate of getter | 98.3 | 100 | 100 | 100 | 93.3 |
| | better/% | | | | | |
| Fuke Qianjin | Number of | 60 | 51 | 52 | 42 | 44 |
| Tablet | people | | | | | |
| (batch 1 of | before | | | | | |
| Embodiment 1) | treatment | | | | | |
| | Significantly | 48 | 49 | 48 | 39 | 36 |
| | reduced | | | | | |
| | Getting | 9 | 2 | 3 | 2 | 7 |
| | better | | | | | |
| | Noneffective | 3 | 0 | 1 | 1 | 1 |
| | Rate of getter | 95 | 100 | 98.1 | 97.4 | 97.7 |
| | better/% | | | | | |
| Fuke Qianjin | Number of | 58 | 52 | 52 | 40 | 43 |
| Tablet | people | | | | | |
| (batch 1 of | before | | | | | |
| Comparative | treatment | | | | | |
| Example) | | | | | | |
| | Significantly | 48 | 48 | 48 | 33 | 32 |
| | reduced | | | | | |
| | Getting | 7 | 3 | 3 | 5 | 8 |
| | better | | | | | |
| | Noneffective | 3 | 1 | 1 | 2 | 3 |
| | Rate of getter | 93.75 | 97.9 | 97.9 | 95 | 90.6 |
| | better/% | | | | | |

It can be seen from Tables 16 to 18 that the Fuke Qianjin Tablets of the present invention is better than the Fuke Qianjin Tablet of Comparative Example 1 in the treatment of chronic pelvic inflammatory disease and adnexitis, wherein the effect of batch 1 of Embodiment is better than that of batch 1, and the effect of batch 1 of Embodiment is better than that of Comparative Example. It reflects that in addition to controlling the content of andrographolide and dehydroandrographolide, it also controls the contents of genistin, Z-ligustilide, and Z-3-butylidenephthalide within a standard range, and the therapeutic effect can be further improved.

### Embodiment 5 Clinical Results of Endometritis

According to the good results shown in the treatment of chronic pelvic inflammatory disease, we also compared the efficacy of the treatment of endometritis. Specifically, 723 patients with endometritis were selected as study subjects, aged between 30 and 40 years old, and the treatment plan was to give antibiotics combined with progesterone for treatment. 0.5 g of metronidazole was added into 250 mL of 0.9% sodium chloride solution, intravenously dripped, once every 8 hours, medroxyprogesterone was taken 4 mg/time, 2 times a day for 14 days after the end of menstruation on the 3rd day, and this is used as a blank control group. The observation group was given the Fuke Qianjin Tablets of Embodiment 1 (3 batches) and Embodiment 2 (3 batches) on the basis of the blank control group, and the control group was given the Fuke Qianjin Tablets prepared in Comparative Example 1 (3 batches) on the basis of the blank control group. The treatment results are shown in Tables 19 to 21.

### Evaluation Criteria

Markedly effective: the clinical symptoms disappeared, the menstruation returned to normal, and the ultrasound examination showed that the inflammation disappeared;

Effective: clinical symptoms got better, and the ultrasound examination showed that the inflammation got better and endometrium was thickened;

Noneffective: no improvement as described above.

TABLE 19
Comparison of clinical efficacy

| | | | | | Markedly | |
|---|---|---|---|---|---|---|
| | | Markedly | | | effective | Effective |
| Batch (number of cases) | | effective | Effective | Noneffective | rate/% | rate/% |
| Embodiment 1 | Batch 1 | 43 | 21 | 7 | 60.56 | 90.14 |
| (216 cases) | (71 cases) | | | | | |
| | Batch 3 | 45 | 21 | 7 | 61.64 | 90.41 |
| | (73 cases) | | | | | |
| | Batch 6 | 45 | 21 | 6 | 62.50 | 91.67 |
| | (72 cases) | | | | | |
| Embodiment 2 | Batch 1 | 48 | 17 | 6 | 67.61 | 91.55 |
| (216 cases) | (71 cases) | | | | | |
| | Batch 3 | 49 | 17 | 6 | 68.06 | 91.67 |
| | (72 cases) | | | | | |
| | Batch 5 | 51 | 17 | 5 | 69.86 | 93.15 |
| | (73 cases) | | | | | |
| Comparative | Batch 1 | 42 | 21 | 9 | 58.33 | 87.50 |
| Example | (72 cases) | | | | | |
| (217 cases) | | | | | | |
| | Batch 2 | 42 | 21 | 10 | 57.53 | 86.30 |
| | (73 cases) | | | | | |
| | Batch 3 | 42 | 22 | 8 | 58.33 | 88.89 |
| | (72 cases) | | | | | |

| | | | | | |
|---|---|---|---|---|---|
| Blank control (74 cases) | 31 | 32 | 16 | 43.24 | 78.38 |

TABLE 20
Comparison of menstruation recovery

| | | Menstrual blood | | | | | |
|---|---|---|---|---|---|---|---|
| | | volume returned | | Menstrual period | | Irregular vaginal | |
| | | to normal | | returned to normal | | bleeding | |
| Batch (number of cases) | | (proportion %) | | (proportion %) | | (proportion %) | |
| Embodiment 1 | Batch 1 | 62 | (87.32%) | 63 | (88.73%) | 4 | (5.63%) |
| (216 cases) | (71 cases) | | | | | | |
| | Batch 3 | 65 | (89.04%) | 65 | (89.04%) | 4 | (5.48%) |
| | (73 cases) | | | | | | |
| | Batch 6 | 65 | (90.28%) | 66 | (91.67%) | 4 | (5.56%) |
| | (72 cases) | | | | | | |
| Embodiment 2 | Batch 1 | 66 | (92.96%) | 66 | (92.96%) | 3 | (4.23%) |
| (216 cases) | (71 cases) | | | | | | |
| | Batch 3 | 67 | (93.06%) | 67 | (93.06%) | 3 | (4.17%) |
| | (72 cases) | | | | | | |
| | Batch 5 | 68 | (93.15%) | 69 | (94.52%) | 3 | (4.11%) |
| | (73 cases) | | | | | | |
| Comparative | Batch 1 | 62 | (86.11%) | 62 | (86.11%) | 5 | (6.94%) |
| Example | (72 cases) | | | | | | |
| (217 cases) | | | | | | | |
| | Batch 2 | 63 | (86.3%) | 63 | (86.3%) | 6 | (8.22%) |
| | (73 cases) | | | | | | |
| | Batch 3 | 62 | (86.11%) | 62 | (86.11%) | 5 | (6.94%) |
| | (72 cases) | | | | | | |

| | | | | | | |
|---|---|---|---|---|---|---|
| Blank control (74 cases) | 38 | (61.29%) | 46 | (62.16%) | 48 | (64.86%) |

TABLE 21
B-ultrasonic examination recovery comparison

| | | | Increased | | Hypogastrium | | Endometrial | |
|---|---|---|---|---|---|---|---|---|
| | | Endometrial | secretion | | bearing-down | | adhesion | |
| Batch (number of cases) | | thickness/mm | (proportion %) | | pain (proportion %) | | (proportion %) | |
| Embodiment 1 | Batch 1 | 6.51 ± 0.46 | 4 | (5.63%) | 6 | (8.45%) | 4 | (5.63%) |
| (216 cases) | (71 cases) | | | | | | | |
| | Batch 3 | 6.65 ± 0.43 | 5 | (6.85%) | 6 | (8.22%) | 4 | (5.48%) |
| | (73 cases) | | | | | | | |
| | Batch 6 | 6.75 ± 0.59 | 4 | (5.56%) | 6 | (8.33%) | 4 | (5.56%) |
| | (72 cases) | | | | | | | |
| Embodiment 2 | Batch 1 | 7.01 ± 0.28 | 3 | (4.23%) | 4 | (5.63%) | 3 | (4.23%) |
| (216 cases) | (71 cases) | | | | | | | |
| | Batch 3 | 7.02 ± 0.41 | 3 | (4.17%) | 5 | (6.94%) | 3 | (4.17%) |
| | (72 cases) | | | | | | | |
| | Batch 5 | 7.18 ± 0.39 | 3 | (4.11%) | 3 | (4.11%) | 3 | (4.11%) |
| | (73 cases) | | | | | | | |
| Comparative | Batch 1 | 5.31 ± 0.57 | 7 | (9.72%) | 9 | (12.5%) | 5 | (6.94%) |
| Example | (72 cases) | | | | | | | |
| (217 cases) | Batch 2 | 5.26 ± 0.42 | 8 | (10.96%) | 9 | (12.33%) | 5 | (6.85%) |
| | (73 cases) | | | | | | | |
| | Batch 3 | 5.18 ± 0.35 | 7 | (9.72%) | 9 | (12.5%) | 5 | (6.94%) |
| | (72 cases) | | | | | | | |

| | | | | | | | |
|---|---|---|---|---|---|---|---|
| Blank control (74 cases) | 4.26 ± 0.34 | 23 | (31.08%) | 22 | (29.73%) | 20 | (27.03%) |

From the data in Tables 19-21, it can be seen that compared to the original Fuke Qianjin Tablets that only control the active ingredient of Herba Andrographis, the Fuke Qianjin Tablets of the present invention that the contents of genistin, and/or Z-ligustilide, and/or Z-3-butylidenephthalide are controlled, the effectiveness of the Fuke Qianjin Tablets in the treatment of endometritis is improved. The specific performance is that the efficiency and the markedly effective rate have been improved, indicating that when the contents of genistin, and/or Z-ligustilide, and/or Z-3-butylidenephthalide are controlled, the Fuke Qianjin Tablets can interact better with antibiotics and progesterone.

From the consistency experiment of the above Embodiments and Comparative Example, it can be seen that in the production process, the detection of the ingredients of the mixed cream sample is added, and the control is within a reasonable range, so that the contents of the five active ingredients in the obtained Fuke Qianjin Tablets can be controlled within a reasonable and narrow range, so that the consistency between batches of the prepared product is better, and the clinical treatment effect is improved.

Finally, it should be noted that the above Embodiments are only used to illustrate the technical solution of the present invention and not to limit the scope of protection of the present invention. For those of ordinary skill in the art, on the basis of the above description and ideas, other different forms of changes or variations can also be made, and it is not necessary and impossible to enumerate all the implementation here. All modifications, equivalent replacements and improvements made within the spirit and principles of the present invention shall be included in the scope of protection claimed in the present invention.

1. A quality control method for Fuke Qianjin Tablets, wherein the quality control method comprises the following steps:
using Radix Et Caulis Flemingiae, Radix Rosa Laevigata, Herba Andrographis, Radix Angelicae Sinensis, Caulis Mahoniae, Zanthoxylum dissitum Hemsl., Caulis Spatholobi and Radix Codonopsis as raw materials;
S1, selecting the Radix Angelicae Sinensis, the Radix Codonopsis, and the Herba Andrographis, crushing into fine powders of 100 meshes or more, respectively, and reserving for use at a powder yield of at least 93.3%;
S2, selecting the Zanthoxylum dissitum Hemsl., the Radix Rosa Laevigata, the Caulis Spatholobi, the Caulis Mahoniae and the Radix Et Caulis Flemingiae, adding water to extract twice, each extraction time being 2 hours, filtering to obtain a filtrate, and concentrating the filtrate into a cream of 1.08/85° C. CF; and
S3, mixing the fine powders of the step S1 and the cream of the step S2 to obtain a mixture, controlling a content of genistin, a content of at least one of Z-ligustilide and Z-3-butylidenephthalide, and a total amount of andrographolide and dehydroandrographolide in the mixture to reach a standard content; and then drying, tabletting and coating to obtain the Fuke Qianjin Tablets.

2. The method according to claim 1, wherein in the step S3, it is controlled that per milligram of the mixture, the content of the genistin is not less than 0.00005 mg, the content of the Z-ligustilide is not less than 0.00394 mg and/or the content of the Z-3-butylidenephthalide is not less than 0.00008 mg, and the content of the andrographolide and the dehydroandrographolide is not less than 0.00619 mg.

3. The method according to claim 2, wherein in the step S3, it is controlled that per milligram of the mixture, the content of the genistin is not less than 0.00007 mg, the content of the Z-ligustilide is not less than 0.0045 mg and/or the content of the Z-3-butylidenephthalide is not less than 0.00011 mg, and the content of the andrographolide and the dehydroandrographolide is not less than 0.00788 mg.

4. The method according to claim 3, wherein it is controlled that per milligram of the mixture, the content of the genistin is 0.00011 mg to 0.00028 mg, the content of the Z-ligustilide is 0.00647 mg to 0.009 mg and/or the content of the Z-3-butylidenephthalide is 0.00023 mg to 0.00045 mg, and the content of the andrographolide and the dehydroandrographolide is not less than 0.01 mg.

5. The method according to claim 2, wherein each of the Fuke Qianjin Tablets prepared by the method contains not less than 0.008 mg of the genistin, not less than 0.7 mg of the Z-ligustilide and/or not less than 0.015 mg of the Z-3-butylidenephthalide, and the total amount of the andrographolide and the dehydroandrographolide is not less than 1.1 mg.

6. The method according to claim 3, wherein each of the Fuke Qianjin Tablets prepared by the method contains not less than 0.012 mg of the genistin, not less than 0.8 mg of the Z-ligustilide and/or not less than 0.02 mg of the Z-3-butylidenephthalide, and the total amount of the andrographolide and the dehydroandrographolide is not less than 1.4 mg.

7. The method according to claim 4, wherein each of the Fuke Qianjin Tablets prepared by the method contains 0.02 mg to 0.05 mg of the genistin, 1.15 mg to 1.65 mg of the Z-ligustilide and/or 0.04 mg to 0.08 mg of the Z-3-butylidenephthalide, and the total amount of the andrographolide and the dehydroandrographolide is not less than 1.93 mg.

8. The method according to claim 1, wherein a detection method adopted in the step S3 is HPLC detection.

9. The method according to claim 8, wherein a preparation process of a test sample described in the step S3 is: taking 0.3 g mixture of the cream and the fine powders, adding 200 mL of 75% formalin to dissolve, weighing, and then undergoing ultrasonic treatment for (15±5) minutes, after the ultrasonic treatment, adding 75% formalin to make up a mass loss, and then taking 2 mL of a dissolving solution and diluting to 10 mL with 75% formalin, passing through a 0.45 μm microporous membrane, and taking the filtrate as a solution to be tested.

10. The method according to claim 1, wherein in the step S3, said controlling makes the contents of the genistin, the Z-ligustilide and/or the Z-3-butylidenephthalide after mixing reach a required range by adjusting an extraction process of the step S2 or a source of the raw materials.

11. Fuke Qianjin Tablets, made of Radix Et Caulis Flemingiae, Caulis Mahoniae, Herba Andrographis, Zanthoxylum dissitum Hemsl., Caulis Spatholobi, Radix Angelicae Sinensis, Radix Codonopsis, and Radix Rosa Laevigata as raw materials, wherein each of the Fuke Qianjin Tablets contains components listed as the below (1), or (2), or (3):
(1) each of the Fuke Qianjin Tablets contains not less than 0.008 mg of genistin, not less than 0.7 mg of Z-ligustilide, and a total amount of andrographolide and dehydroandrographolide is not less than 1.1 mg;
(2) each of the Fuke Qianjin Tablets contains not less than 0.008 mg of genistin, not less than 0.015 mg of Z-3-butylidenephthalide, and a total amount of the andrographolide and the dehydroandrographolide is not less than 1.1 mg;
(3) each of the Fuke Qianjin Tablets contains not less than 0.008 mg of genistin, not less than 0.7 mg of Z-ligustilide, not less than 0.015 mg of Z-3-butylidenephthalide, and a total amount of andrographolide and dehydroandrographolide is not less than 1.1 mg.

12. (canceled)

13. The Fuke Qianjin Tablets according to claim 11, wherein each of the Fuke Qianjin Tablets contains components listed as the below (1), or (2), or (3):
(1) each of the Fuke Qianjin Tablets contains not less than 0.012 mg of genistin, not less than 0.8 mg of Z-ligustilide, and a total amount of andrographolide and dehydroandrographolide is not less than 1.4 mg;
(2) each of the Fuke Qianjin Tablets contains not less than 0.012 mg of genistin, not less than 0.02 mg of Z-3-butylidenephthalide, and a total amount of the andrographolide and the dehydroandrographolide is not less than 1.4 mg;
(3) each of the Fuke Qianjin Tablets contains not less than 0.012 mg of genistin, not less than 0.8 mg of Z-ligustilide, not less than 0.02 mg of Z-3-butylidenephthalide, and a total amount of andrographolide and dehydroandrographolide is not less than 1.4 mg.

14. The Fuke Qianjin Tablets according to claim 13, wherein each of the Fuke Qianjin Tablets contains components listed as the below (1), or (2), or (3):
(1) each of the Fuke Qianjin Tablets contains 0.02 mg to 0.05 mg of genistin, 1.15 mg to 1.65 mg of Z-ligustilide, and a total amount of andrographolide and dehydroandrographolide is not less than 1.93 mg;
(2) each of the Fuke Qianjin Tablets contains 0.02 mg to 0.05 mg of genistin, 0.04 mg to 0.08 mg of Z-3-butylidenephthalide, and a total amount of the andrographolide and the dehydroandrographolide is not less than 1.93 mg;
(3) each of the Fuke Qianjin Tablets contains 0.02 mg to 0.05 mg of genistin, 1.15 mg to 1.6 mg of Z-ligustilide, 0.04 mg to 0.08 mg of Z-3-butylidenephthalide, and a total amount of andrographolide and dehydroandrographolide is not less than 1.93 mg.

15. (canceled)

16. The Fuke Qianjin Tablets according to claim 11, wherein the contents of the genistin, the Z-ligustilide and the Z-3-butylidenephthalide, and the total amount of the andrographolide and the dehydroandrographolide are determined by HPLC detection.

17. The Fuke Qianjin Tablets according to claim 16, wherein a test sample for the HPLC detection is prepared by the following method: taking 10 Fuke Qianjin Tablets, removing coating, accurately weighing, porphyrizing, taking 0.3 g, placing in a container, accurately adding 20 mL of 75% formalin, ultrasonically extracting for (15±5) minutes, after cooling to room temperature, using 75% formalin to make up a mass loss, passing through a 0.45 μm microporous membrane, and taking the filtrate as a solution to be tested.

18. The Fuke Qianjin Tablets according to claim 16, wherein the HPLC detection is performed according to the following conditions: using Kromasil 100-5-C18 chromatographic column (250 mm×4.6 mm, 5 μm), with mobile phases using acetonitrile as an A phase and 0.1% phosphoric acid aqueous solution as a B phase, gradient eluting, with a flow rate being 1.0 mL·min−1, a detection wavelength being 254 nm, a column temperature being (30±0.5)° C., and an injection volume being 10 μL.

19. The Fuke Qianjin Tablets according to claim 11, wherein dosage of the Radix Angelicae Sinensis, the Radix Codonopsis, the Herba Andrographis and the Zanthoxylum dissitum Hemsl. is each 9% of a total amount of medicinal materials; and dosage of the Radix Rosa Laevigata, the Caulis Spatholobi, the Caulis Mahoniae and the Radix Et Caulis Flemingiae is each 16% of the total amount of the medicinal materials.

20. Use of the Fuke Qianjin Tablets according to claim 11 in preparing treatment for a gynecological disease.

21. The method according to claim 9, wherein conditions of the HPLC detection in the step S3 are: using Kromasil 100-5-C18 chromatographic column (250 mm×4.6 mm, 5 μm), with mobile phases using acetonitrile as an A phase and 0.1% phosphoric acid aqueous solution as a B phase, gradient eluting, with a flow rate being 1.0 mL·min−1, a detection wavelength being 254 nm, a column temperature being (30±0.5)° C., and an injection volume being 10 μL.
